(19) United States
(12) Patent Application Publication
(10) Pub. No.: US 2026/0331216 A1
(43) Pub. Date: Oct. 22, 2026

(54) METHOD FOR ENHANCING CHROMATIN INTERACTION INFORMATION BASED ON RNA-DNA AND DNA-DNA INTERACTION INFORMATION

(71) Applicant: Institute of Basic Medical Sciences (IBMS) of the Chinese Academy of Medical Sciences (CAMS), Beijing (CN)

(72) Inventors: Yang CHEN, Beijing (CN); Yurong YANG, Beijing (CN); Minghong LI, Beijing (CN); Xueyuan LI, Beijing (CN)

(73) Assignee: Institute of Basic Medical Sciences (IBMS) of the Chinese Academy of Medical Sciences (CAMS), Beijing (CN)

(21) Appl. No.: 19/682,834

(22) Filed: May 20, 2026

(30) Foreign Application Priority Data
May 21, 2025 (CN) 202510656424.1

(51) Int. Cl. G16B 20/00; G06N 3/0464; G06N 3/08; G16B 40/00

(52) U.S. Cl. CPC G16B 20/00; G06N 3/0464; G06N 3/08; G16B 40/00

(57) ABSTRACT

A method for enhancing chromatin interaction information based on RNA-DNA and DNA-DNA interaction information includes the following steps: collecting iMARGI data of RNA-DNA interactions and raw Hi-C data of DNA-DNA interactions from the same cell line; performing feature extraction on the iMARGI data, and then calculating a Pearson correlation coefficient, to obtain a genome-wide DNA-DNA feature correlation coefficient matrix integrating RNA-DNA interaction information; performing downsampling on the raw Hi-C data, and then performing FAN completion processing to obtain a completed Hi-C matrix; constructing a CNN network model, taking the DNA-DNA feature correlation coefficient matrix integrating RNA-DNA interaction information and the completed Hi-C matrix as inputs of the model, and training the model; and for the trained model, inputting data of different RNA types to predict a corresponding Hi-C matrix for downstream analysis.

### CROSS-REFERENCE TO RELATED APPLICATION

The present application claims priority to Chinese Patent Application No. 202510656424.1, filed on May 21, 2025, the disclosure of which is incorporated herein by reference in its entirety.

### TECHNICAL FIELD

The present disclosure relates to the technical field of bioinformatics, and in particular, to a method for enhancing chromatin interaction information based on Ribonucleic Acid (RNA)-Deoxyribonucleic Acid (DNA) and DNA-DNA interaction information.

### BACKGROUND

In recent years, research on the functions of RNA in the nucleus of eukaryotic cells and their interactions with chromatin has been gradually deepened. RNA is no longer regarded merely as an intermediary of gene expression; it plays diverse and important roles in the dynamic regulation of chromatin conformation. Various types of RNA exist in cells, including coding RNAs and non-coding RNAs (such as long non-coding RNA (lncRNA), small nucleolar RNA (snoRNA), small nuclear RNA (snRNA), etc.). These RNAs participate in gene transcription, epigenetic regulation, and cell fate determination by regulating the structure and function of chromatin. Research on RNA-chromatin interactions provides a new perspective for understanding the regulation of gene expression.

Currently, RNA-chromatin interactions are considered to play an important role in the organization within the cell nucleus. By binding to chromatin, RNA can regulate the open state of chromatin, affect gene transcription programs, and ensure that cells maintain normal gene expression patterns during physiological processes. Existing genomic research techniques, such as High-throughput Chromosome Conformation Capture (Hi-C) and Enhanced Cross-Linking Immunoprecipitation Sequencing (eCLIP-seq), have been able to provide preliminary data on chromatin-RNA interactions. However, most existing techniques focus on analyzing the three-dimensional structure of DNA and lack tools for integrating RNA interaction information, making it impossible to fully reveal the role of RNA in chromatin regulation.

In related technologies, Mapping of RNA-Genome Interaction (MARGI) technology, as an early method for studying RNA-DNA interactions, combines cross-linked immunoprecipitation with high-throughput sequencing, and can initially analyze RNA binding sites on the genome. However, this method still has certain limitations in spatial accuracy and resolution. The subsequently developed in situ Mapping of RNA-Genome Interaction (iMARGI) technology has achieved significant improvements in in situ capture and spatial resolution, allowing more accurate localization of RNA-DNA interaction sites and experiments under conditions that maintain chromatin structure, thereby enhancing its biological significance.

Global RNA Interaction with DNA (GRID-seq), as a deep sequencing technology, can capture global RNA-DNA interactions, revealing chromatin-associated RNA sites and their specific binding positions on chromatin. This technology provides an important tool for studying the role of RNA in chromatin regulation. However, GRID-seq currently cannot fully process multimodal interaction data between different RNA types and chromatin, and lacks the ability to integrate with other genomic data (such as Hi-C and epigenetic data).

RNA in situ conformation sequencing (RIC-seq) provides a deeper understanding of the spatial organization of RNA and chromatin by measuring the secondary and tertiary structures of RNA and RNA-RNA interactions at high resolution. Although RIC-seq can provide rich information on RNA structure, the complexity of data interpretation and how to integrate these data with other chromatin interaction data remain technical challenges.

To fill these gaps, RNA & DNA Split-Pool Recognition of Interactions by Tag Extension (RD-SPRITE) technology maps the spatial interactions between RNA and DNA into high-resolution maps, helping to reveal the role of RNA in the spatial regulation of chromatin. However, although the above technologies provide rich tools and data sources for the study of RNA-chromatin interactions, due to the diversity of technical workflows, there is still a lack of a systematic computational analysis framework that can effectively integrate RNA interaction data produced by different technologies with chromatin conformation data such as Hi-C. Therefore, developing an efficient analysis tool capable of integrating RNA interaction data with Hi-C data has become an urgent problem to be solved in the current field of bioinformatics.

### SUMMARY

To address the above technical problems, an objective of the present disclosure is to provide a method for enhancing chromatin interaction information based on RNA-DNA and DNA-DNA interaction information. The method integrates RNA interaction data with Hi-C data, constructs a multi-modal data integration and analysis platform, which can compensate for the deficiencies of existing technologies in multi-modal data integration, and promote further development of research on RNA-chromatin interactions.

To achieve the above technical objective, the present disclosure provides the following technical solution:

A method for enhancing chromatin interaction information based on RNA-DNA and DNA-DNA interaction information, including the following steps:
S1, collecting iMARGI data of RNA-DNA interactions and raw Hi-C data of DNA-DNA interactions from the same cell line;
S2, performing feature extraction on the iMARGI data, and then calculating a Pearson correlation coefficient, to obtain a genome-wide DNA-DNA feature correlation coefficient matrix integrating RNA-DNA interaction information; performing downsampling on the raw Hi-C data, and then performing Filtering by Adaptive Normalization (FAN) completion processing to obtain a completed Hi-C matrix;
S3, constructing a Convolutional Neural Network (CNN) network model, taking the DNA-DNA feature correlation coefficient matrix integrating RNA-DNA interaction information and the completed Hi-C matrix as inputs of the model, and training the model; and
S4, for the trained model, inputting data of different RNA types to predict a corresponding Hi-C matrix for downstream analysis.

In an embodiment, step S1 specifically includes: obtaining the iMARGI data and the raw Hi-C data from public databases or experimental data; performing quality control on the obtained raw data to remove noisy data.

In an embodiment, step S2 specifically includes:
performing feature extraction on the iMARGI data of RNA-DNA interactions, and performing Pearson correlation coefficient calculation on the extracted features, to obtain the DNA-DNA feature correlation coefficient matrix integrating RNA-DNA interaction information;
performing Knight-Ruiz (KR) normalization processing on the raw Hi-C data, performing smoothing processing on the KR-normalized Hi-C data using downsampling rates of 1/5 and 1/10, and performing completion processing on the downsampled Hi-C data using a FAN method to obtain the completed Hi-C matrix.

In an embodiment, in step S3, the inputs of the model include two parts: the DNA-DNA feature correlation coefficient matrix integrating RNA-DNA interaction information and the completed Hi-C matrix;
step S3 further includes:
performing sample division in the following manner: dividing the DNA-DNA feature correlation coefficient matrix integrating RNA-DNA interaction information and the completed Hi-C matrix into n K×K dimensional sub-matrices as input samples; and
dividing the input samples into a training set, a validation set, and a test set, which are respectively used for training, validation, and evaluation of the model.

In an embodiment, the constructing a CNN network model in step S3 includes:
for the completed Hi-C matrix, performing downsampling twice using a Double_Conv module combined with a DownConv layer, performing upsampling twice using the Double_Conv module combined with an UpConv layer, and finally obtaining a first result via the Double_Conv module;
for the DNA-DNA feature correlation coefficient matrix integrating RNA-DNA interaction information, obtaining a second result using the Double_Conv module three times;
concatenating the first result and the second result, and then obtaining a final enhanced Hi-C matrix using the Double_Conv module twice.

In an embodiment, the training the model in step S3 includes:
taking the DNA-DNA feature correlation coefficient matrix integrating RNA-DNA interaction information and the completed Hi-C matrix as inputs of the model, extracting multi-scale feature vectors from the two matrices respectively via a convolutional neural network, concatenating the two obtained vectors, and restoring a predicted Hi-C matrix corresponding to an input size of a target Hi-C matrix through deconvolution operations;
measuring an error between the predicted Hi-C matrix and a ground-truth Hi-C matrix using Least Absolute Deviations Loss (L1 loss) and perceptual loss, and iteratively optimizing model parameters based on the error.

In an embodiment, the measuring an error between the predicted Hi-C matrix and a ground-truth Hi-C matrix using L1 loss and perceptual loss, and iteratively optimizing model parameters based on the error specifically includes:
combining the L1 loss and the perceptual loss to optimize model weights, to handle data imbalance and improve model performance on sparse data;
training the model using an Adaptive Moment Estimation (Adam) optimizer, and performing cross-validation and parameter tuning to improve generalization ability of the model.

In an embodiment, step S3 further includes:
for multiple models trained by taking DNA-DNA feature correlation coefficient matrices integrating different types of RNA-DNA interaction information as input sources, inputting preprocessed RNA data into the multiple trained models respectively, so that the multiple models respectively output predicted chromatin interaction enhancement matrices; verifying prediction accuracy and stability of the trained models by comparing the enhancement matrices with a ground-truth Hi-C matrix.

In an embodiment, in step S4, the downstream analysis includes: Hi-C chromatin loop analysis mediated by different RNA types, validation using Cross-Linking Immunoprecipitation Sequencing (CLIP-seq) data and Chromatin Immunoprecipitation Sequencing (ChIP-seq) data, enrichment of chromatin loops in subnuclear structures and Enhancer-Promoter (EP) sites, effects of different RNA types on chromatin hierarchical structures, exploration of effects of RNA on chromatin conformation via model attribution, exploration of effects of RNA on TAD boundaries and chromatin loop anchors via model attribution, and visualization analysis of RNA and higher-order chromatin structures.

In an embodiment, in the downstream analysis, the Hi-C chromatin loop analysis mediated by different RNA types includes: identifying chromatin loops on raw Hi-C data and predicted Hi-C data using a Mustache tool, and performing Venn analysis on the chromatin loops of the raw data and the chromatin loops of the predicted data;
the validation using CLIP-seq data and ChIP-seq data includes: evaluating, using CLIP-seq data, whether, near chromatin loop anchors mediated by different RNA types, there is specific co-localization with RNA-binding proteins (RBPs) bound to nascent RNA; analyzing, in combination with public ChIP-seq data, whether chromatin loop anchors in predicted results are enriched for additional chromatin regulatory factors;
the enrichment of chromatin loops in subnuclear structures and EP sites includes: respectively calculating enrichment of Hi-C data predicted by different types of RNA in subnuclear structures and EP sites, revealing that lncRNA frequently participates in construction of promoter-enhancer interactions and that snRNA and snoRNA play roles in nucleolus and nuclear speckle regions;
the effects of different RNA types on chromatin hierarchical structures include: respectively calculating effects of different types of RNA on chromatin loops, Topologically Associating Domains (TADs), and A/B compartments of Hi-C data, revealing that lncRNA participates in local fine regulation, while snRNA/snoRNA has advantages in long-range interactions and larger-scale structural remodeling.

In an embodiment, in the downstream analysis, the exploration of effects of RNA on chromatin conformation via model attribution includes: calculating contribution features of lncRNA, messenger RNA (mRNA), snRNA, and snoRNA at TAD boundaries and chromatin loop anchors; revealing a promoting effect of RNA on chromatin loop structures and negative or neutral regulation at TAD boundaries; indicating that the effects of RNA on chromatin conformation are scale-dependent and tend to mediate small-scale, specific chromatin loop structures;
the exploration of effects of RNA on TAD boundaries and chromatin loop anchors via model attribution includes: calculating contribution features of lncRNA, mRNA, snRNA, and snoRNA at TAD boundaries and chromatin loop anchors; and displaying interaction distribution maps with positional information for chromatin interactions at a given locus affected and unaffected by RNA.

### BRIEF DESCRIPTION OF THE DRAWINGS

In order to more clearly illustrate the technical solutions in the embodiments of the present disclosure, the following briefly introduces the accompanying drawings used in the description of the embodiments. Obviously, the accompanying drawings described below are only some embodiments of the present disclosure. For those of ordinary skill in the art, other accompanying drawings can be obtained from these drawings without creative efforts.

FIG. 1 is a schematic flow chart of a method for enhancing chromatin interaction information based on RNA-DNA and DNA-DNA interaction information provided by an embodiment of the present disclosure.

FIG. 2 is a schematic diagram of correlation coefficient matrix calculation provided by an embodiment of the present disclosure.

FIG. 3 is a schematic diagram of sample division provided by an embodiment of the present disclosure.

FIG. 4 is a schematic structural diagram of a network model provided by an embodiment of the present disclosure.

FIG. 5 is a graph showing a variation curve of a model loss function with training epochs provided by an embodiment of the present disclosure.

FIG. 6 is a schematic diagram showing overlap between Hi-C prediction results of four RNA types and chromatin loops of raw data provided by an embodiment of the present disclosure.

FIG. 7 is a clustered heatmap of co-localization of ChIP-seq data of different epigenetic signals at chromatin loop anchors in raw data and Hi-C data predicted by four RNA types provided by an embodiment of the present disclosure.

FIG. 8 is a clustered heatmap of co-localization of different CLIP-seq data at chromatin loop anchors in raw Hi-C data and Hi-C data predicted by four RNA types provided by an embodiment of the present disclosure.

FIG. 9 is a schematic diagram showing enrichment in subnuclear structures and EP enrichment provided by an embodiment of the present disclosure.

FIG. 10 is a schematic diagram showing distribution characteristics of Hi-C data of different RNA types at different scales provided by an embodiment of the present disclosure.

FIG. 11 is a schematic diagram showing the effect of RNA on TAD boundaries provided by an embodiment of the present disclosure.

FIG. 12 is a schematic diagram showing the effect of RNA on chromatin loop anchors provided by an embodiment of the present disclosure.

FIG. 13 is a schematic diagram showing the position distribution of the effect of RNA on TAD boundary interactions provided by an embodiment of the present disclosure.

FIG. 14 is a schematic diagram showing the position distribution of the effect of RNA on chromatin loop anchors provided by an embodiment of the present disclosure.

FIG. 15 is a schematic diagram of multi-omics visualization of the effects of lncRNA on the SET And MYND Domain Containing 3 (SMYD3) gene locus: ChIP-seq co-localization provided by an embodiment of the present disclosure.

FIG. 16 is a schematic diagram of multi-omics visualization of the effects of lncRNA on the SMYD3 locus: CLIP-seq co-localization provided by an embodiment of the present disclosure.

### DETAILED DESCRIPTION OF THE EMBODIMENTS

In order to make the objectives, technical solutions, and advantages of the embodiments of the present disclosure clearer, the technical solutions in the embodiments of the present disclosure will be described clearly and completely below in conjunction with the accompanying drawings of the embodiments of the present disclosure. Apparently, the described embodiments are part of the embodiments of the present disclosure, rather than all of the embodiments. Based on the described embodiments of the present disclosure, all other embodiments obtained by those of ordinary skill in the art without creative effort shall fall within the protection scope of the present disclosure.

An embodiment of the present disclosure provides a method for enhancing chromatin interaction information based on RNA-DNA and DNA-DNA interaction information. The method implements a deep learning-based prediction model (HiClip model) capable of inferring RNA-mediated Hi-C maps from RNA-DNA interaction data and Hi-C data.

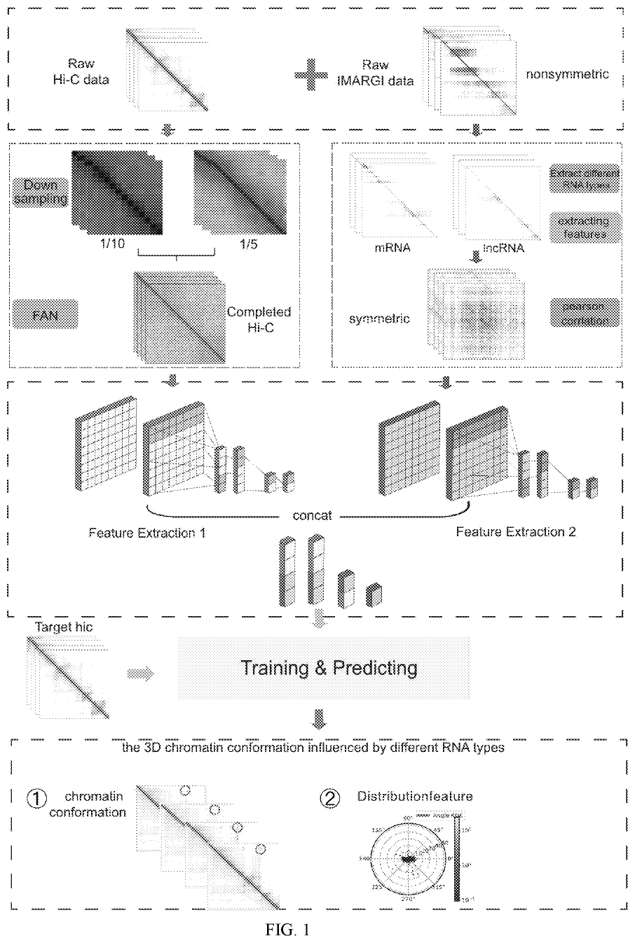

Referring to FIG. 1, the method includes the following steps:

S1, collecting iMARGI data of RNA-DNA interactions and raw Hi-C data of DNA-DNA interactions from the same cell line.

In this step, the iMARGI data and the raw Hi-C data can be obtained from public databases or experimental data, and quality control is performed on the obtained raw data to remove noisy data and low-quality data.

S2, performing feature extraction on the iMARGI data, and then calculating a Pearson correlation coefficient, to obtain a genome-wide DNA-DNA feature correlation coefficient matrix integrating RNA-DNA interaction information; performing downsampling on the raw Hi-C data, and then performing FAN completion processing to obtain a completed Hi-C matrix.

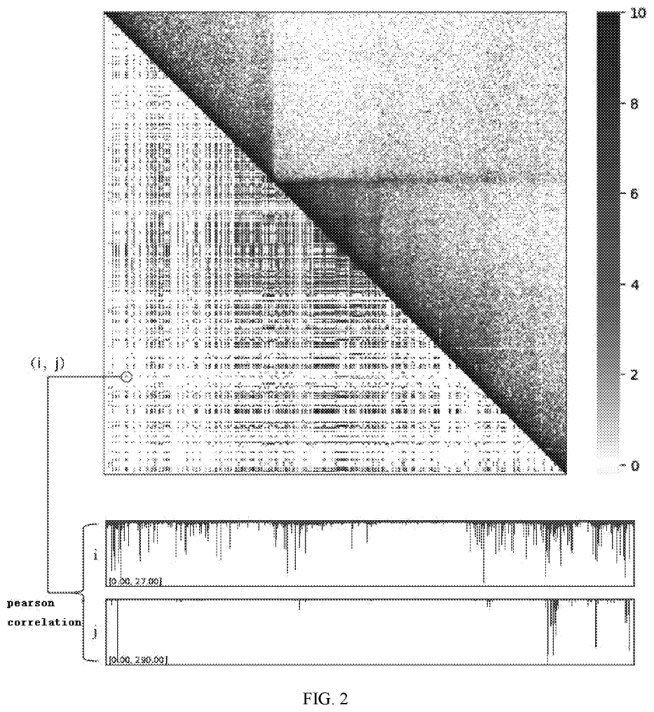

Specifically, referring to FIG. 2, feature extraction is performed on the RNA-DNA interaction data (iMARGI data), and Pearson correlation coefficient calculation is performed on the extracted features to obtain the DNA-DNA feature correlation coefficient matrix integrating RNA-DNA interaction information.

Knight-Ruiz (KR) normalization processing is performed on the raw Hi-C data to eliminate technical noise and batch effects; smoothing processing is performed on the KR-normalized Hi-C data using downsampling rates of 1/5 and 1/10 to ensure that sufficient features are captured from low-resolution matrices; completion processing is performed on the downsampled Hi-C data using a FAN method (Framework for the Analysis of Hi-C data) to obtain the completed Hi-C matrix.

It should be noted that the Hi-C data, Hi-C matrix, and Hi-C map mentioned in the embodiments of the present disclosure can sometimes be used interchangeably, and their intended meanings are consistent when the differences therebetween are not emphasized.

S3, constructing a CNN network model, taking the DNA-DNA feature correlation coefficient matrix integrating RNA-DNA interaction information and the completed Hi-C matrix as inputs of the model, training the model, and outputting an enhanced Hi-C matrix.

The inputs of the CNN network model include two parts: the DNA-DNA feature correlation coefficient matrix integrating RNA-DNA interaction information and the completed Hi-C matrix.

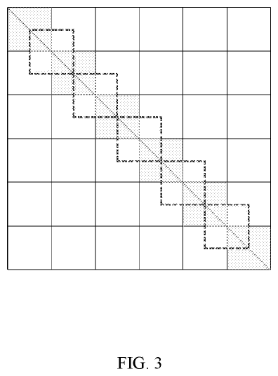

The method further includes: performing sample division in the following manner: dividing the DNA-DNA feature correlation coefficient matrix integrating RNA-DNA interaction information and the completed Hi-C matrix into n K×K dimensional sub-matrices as input samples, referring to FIG. 3; and dividing the input samples into a training set, a validation set, and a test set, which are respectively used for training, validation, and evaluation of the model. During model training, the data in the training set is used for iterative fitting; during the training process, the data in the validation set is used to evaluate the training effect of the model and whether there is a tendency to overfit. Finally, after the model training is completed, the data in the test set is used to evaluate the overall performance of the model.

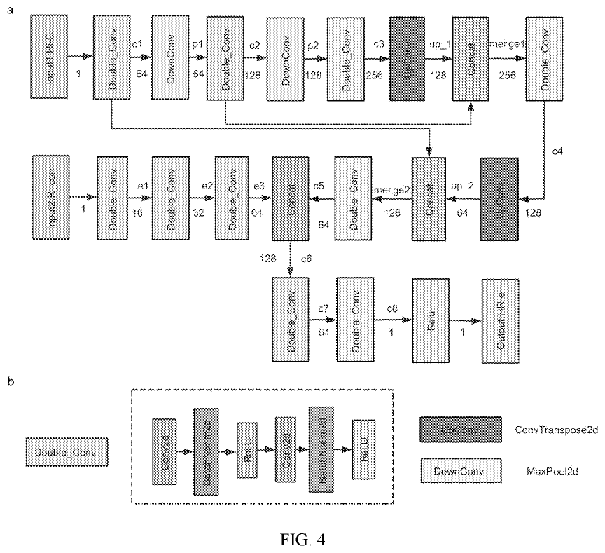

The structure of the model is shown in FIG. 4. Constructing the CNN network model includes: for the completed Hi-C matrix, performing downsampling twice using a Double_Conv module combined with a DownConv layer, performing upsampling twice using the Double_Conv module combined with an UpConv layer, and finally obtaining a first result c5 via the Double_Conv module; for the DNA-DNA feature correlation coefficient matrix (R_corr) integrating RNA-DNA interaction information, obtaining a second result e3 using the Double_Conv module three times; concatenating the first result c5 and the second result e3, and then obtaining a final enhanced Hi-C matrix using the Double_Conv module twice.

Training the CNN network model constructed above includes: taking the DNA-DNA feature correlation coefficient matrix integrating RNA-DNA interaction information and the completed Hi-C matrix as inputs of the model, extracting multi-scale feature vectors from the two matrices respectively via a convolutional neural network, concatenating the two obtained vectors, and restoring a predicted Hi-C matrix corresponding to an input size of a target Hi-C matrix (i.e., the enhanced Hi-C matrix) through deconvolution operations; measuring an error between the predicted Hi-C matrix and a ground-truth Hi-C matrix (i.e., the target hic in FIG. 1) using Least Absolute Deviations Loss (L1 loss) and perceptual loss, and iteratively optimizing model parameters based on the error.

Specifically, the L1 loss and the perceptual loss are combined to optimize model weights to handle data imbalance and improve model performance on sparse data; an Adaptive Moment Estimation (Adam) optimizer is used to train the model, and cross-validation and parameter tuning are performed to improve generalization ability of the model. The L1 loss calculates the sum of absolute differences between the predicted Hi-C matrix and the ground-truth Hi-C matrix, to supervise pixel-level reconstruction accuracy and adapt to the sparsity of Hi-C data. The perceptual loss extracts high-level features through a pre-trained network to evaluate the spatial structure similarity of the predicted matrix. Since the Hi-C matrix contains numerous weak contact values and few strong contact values, using L1 loss avoids over-attention to outliers and stabilizes model training. Meanwhile, by element-wise comparison of contact values against a ground-truth value, the L1 loss enables the overall contact intensity value distribution of the matrix obtained after model training to be more reasonable. The perceptual loss not only compares point-by-point differences of the matrix, but first maps the input matrix to a higher-level feature representation via a feature extraction network, and then compares feature differences. This means that the perceptual loss can help the model reduce over-smoothing and preserve macroscopic structures, supporting subsequent downstream analysis (e.g., identifying structures such as TADs, loops, etc.). By jointly optimizing the two losses, the model can simultaneously ensure the accuracy of single-point contact intensity and the integrity of macroscopic structures such as chromatin loops and TAD boundaries.

After the above training is completed, the model weights are fixed. To evaluate the performance and generalization ability of the trained model, the method of this embodiment further performs prediction and verification on the model. Prediction and verification of the model include: for multiple models trained by taking DNA-DNA feature correlation coefficient matrices integrating different types of RNA-DNA interaction information as input sources, inputting preprocessed RNA data into the multiple trained models respectively, so that the multiple models respectively output predicted chromatin interaction enhancement matrices, i.e., enhanced Hi-C matrices; verifying the prediction accuracy and stability of the trained models by comparing the enhancement matrices output by the models with the ground-truth Hi-C matrix.

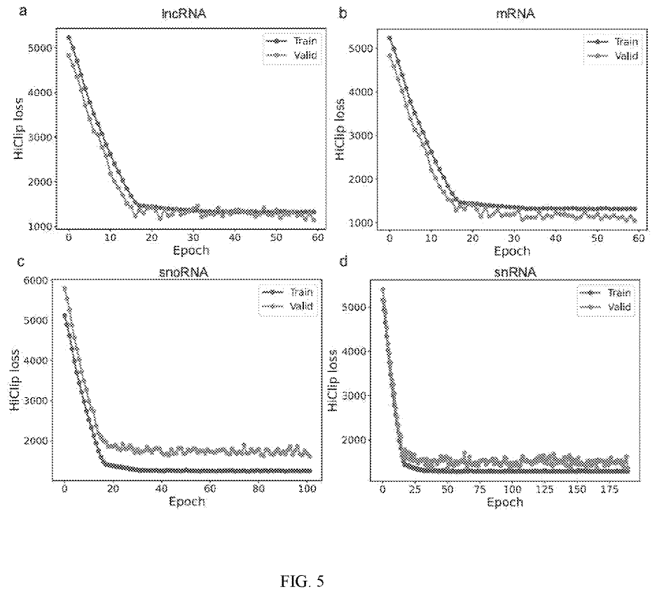

The preprocessed RNA data refers to the DNA-DNA feature correlation coefficient matrices integrating RNA-DNA interaction type information. Experimental results on data of different RNA types are shown in FIG. 5; cross-validation and repeated experiments ensure that the model has good stability and accuracy.

S4, for the trained model, inputting data of different RNA types to predict a corresponding Hi-C matrix for downstream analysis.

In the embodiments of the present disclosure, the data of different RNA types refers to, separately obtaining, for each type of RNA, information on the interaction of that type of RNA with the whole genome, and then converting it into a DNA-DNA feature matrix. The downstream analysis includes: Hi-C chromatin loop analysis mediated by different RNA types, validation using CLIP-seq data and ChIP-seq data, enrichment of chromatin loops in subnuclear structures and EP sites, effects of different RNA types on chromatin hierarchical structures, exploration of the effects of RNA on chromatin conformation via model attribution, exploration of effects of RNA on TAD boundaries and chromatin loop anchors via model attribution, and visualization analysis of RNA and higher-order chromatin structures.

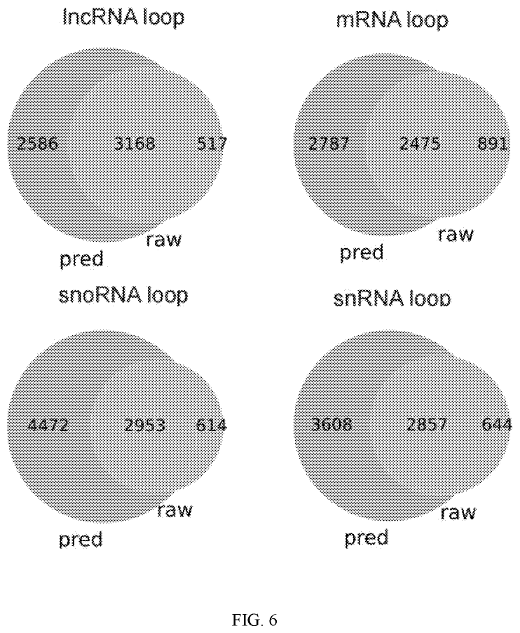

The Hi-C chromatin loop analysis mediated by different RNA types includes: identifying chromatin loops on the raw Hi-C data and the predicted Hi-C data using the Mustache tool, and performing Venn analysis on the chromatin loops from the raw data and the predicted data, as shown in FIG. 6.

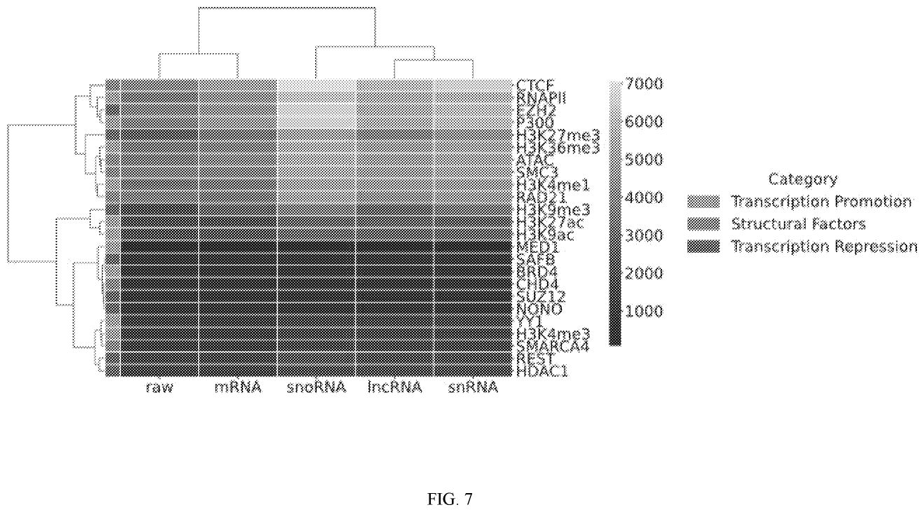

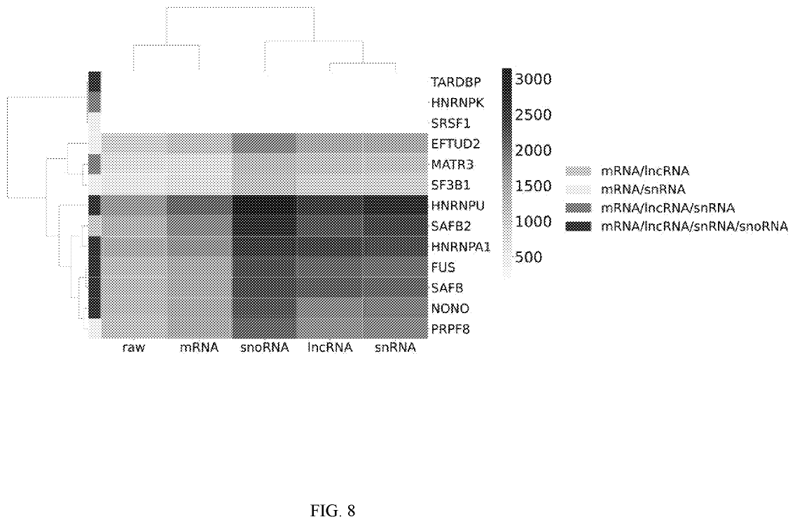

The validation using CLIP-seq data and ChIP-seq data includes: evaluating, using CLIP-seq data, whether, near chromatin loop anchors mediated by different RNA types, there is specific co-localization with RNA-binding proteins (RBPs) bound to nascent RNA; and combining public ChIP-seq data to analyze whether chromatin loop anchors in predicted results are enriched for additional chromatin regulatory factors (e.g., transcription factors, histone modification marks) to further verify the accuracy and reliability of the model, as shown in FIGS. 7 and 8.

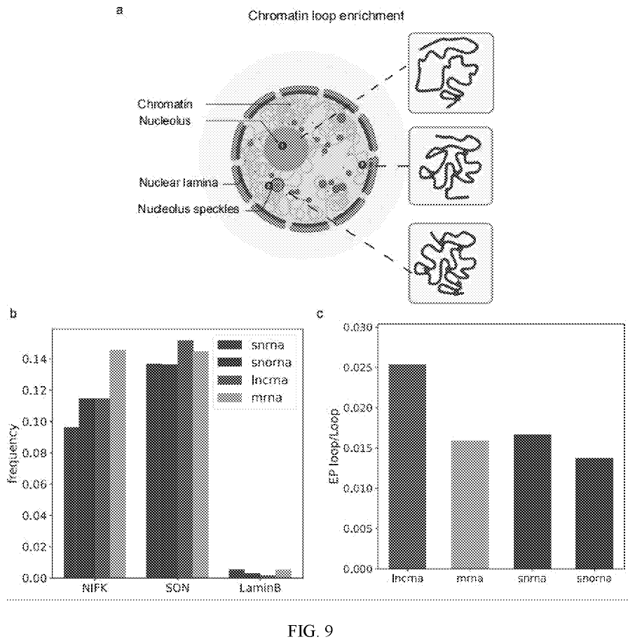

The enrichment of chromatin loops in subnuclear structures and EP sites includes: respectively calculating the enrichment of Hi-C data predicted by different types of RNA in subnuclear structures and EP sites, revealing that lncRNA frequently participates in the construction of promoter-enhancer interactions and that snRNA and snoRNA play important roles in nucleoli and nuclear speckle regions, as shown in FIG. 9.

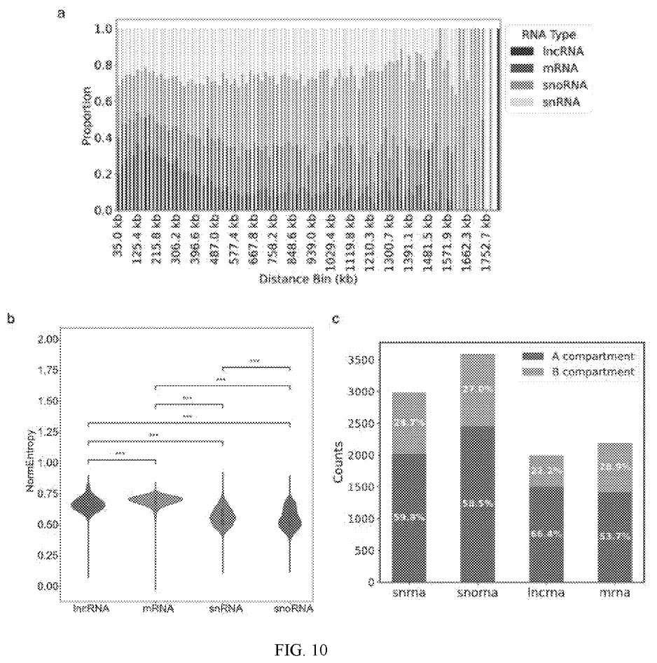

The effects of different RNA types on chromatin hierarchical structures include: respectively calculating effects of different types of RNA on chromatin loops, Topologically Associating Domains (TADs), and A/B compartments of Hi-C data, revealing that lncRNA mainly participates in local fine regulation, whereas snRNA/snoRNA shows clear advantages in long-range interactions and larger-scale structural remodeling, as shown in FIG. 10.

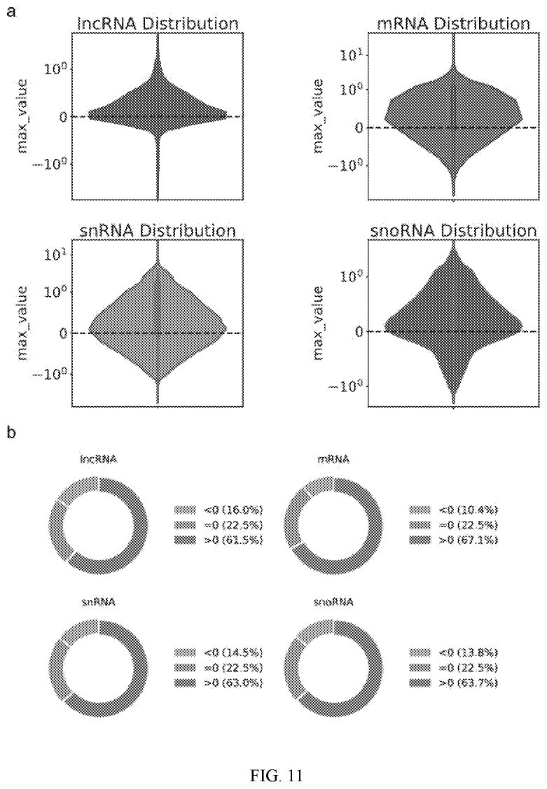

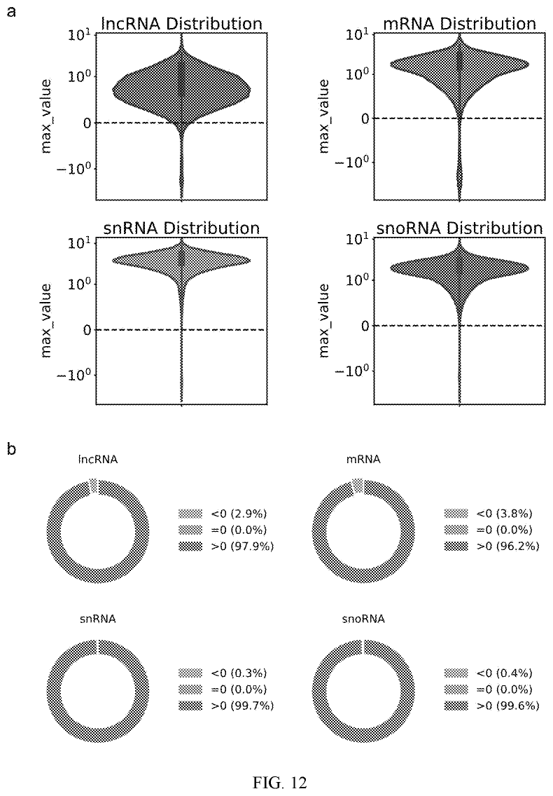

The present disclosure further provides a model attribution module. Specifically, the exploration of the effects of RNA on chromatin conformation via model attribution includes: calculating the contribution features of lncRNA, mRNA, snRNA, and snoRNA at TAD boundaries and chromatin loop anchors; revealing a promoting effect of RNA on chromatin loop structures and certain negative or neutral regulation at TAD boundaries; indicating that the effects of RNA on chromatin conformation are scale-dependent and tend to mediate small-scale, specific chromatin loop structures, as shown in FIGS. 11 and 12.

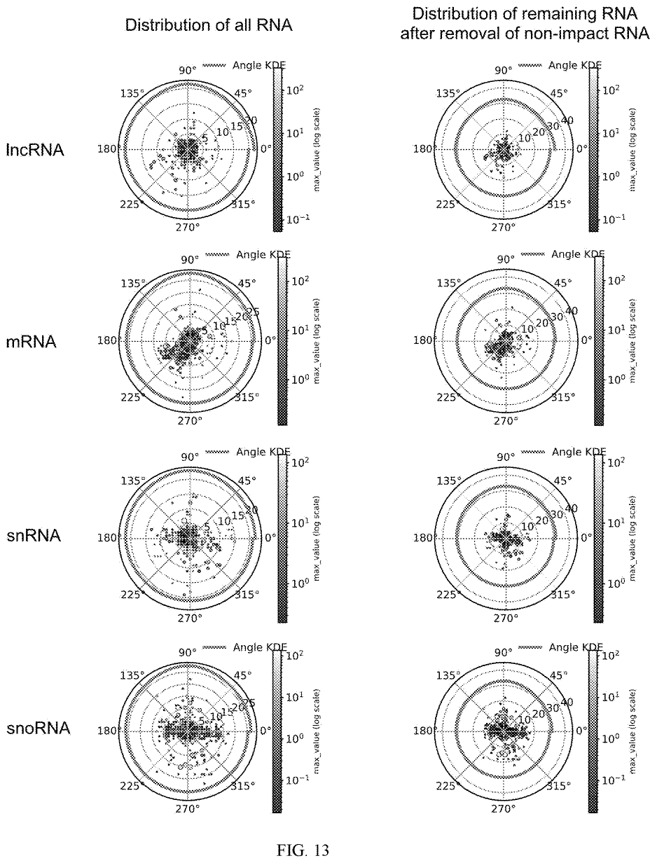

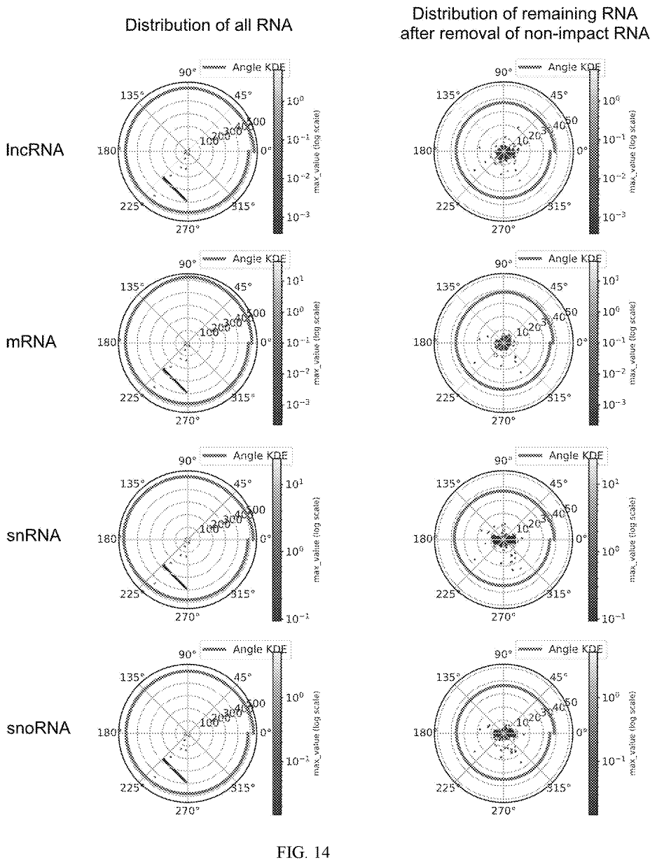

Exploration of the effects of RNA on TAD boundaries and chromatin loop anchors via model attribution includes: calculating the contribution features of lncRNA, mRNA, snRNA, and snoRNA at TAD boundaries and chromatin loop anchors; and displaying interaction distribution maps with positional information for chromatin interactions at a given locus affected and unaffected by RNA. As shown in FIGS. 13 and 14, TAD boundaries are unaffected by RNA at many positions, which are distributed in the lower-left portion of the chromatin interaction locus and within the genomic region of the chromatin interaction, whereas chromatin loop anchors are less frequently unaffected by RNA.

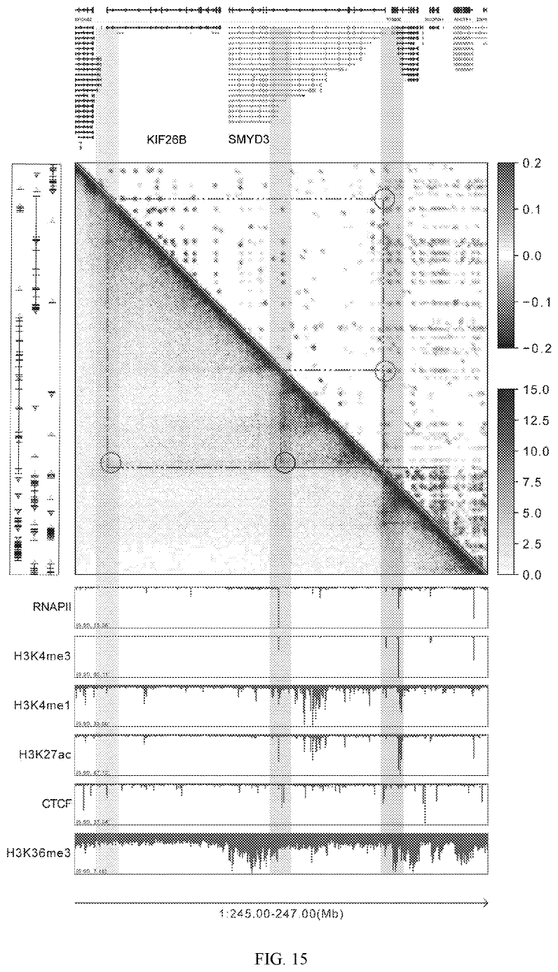

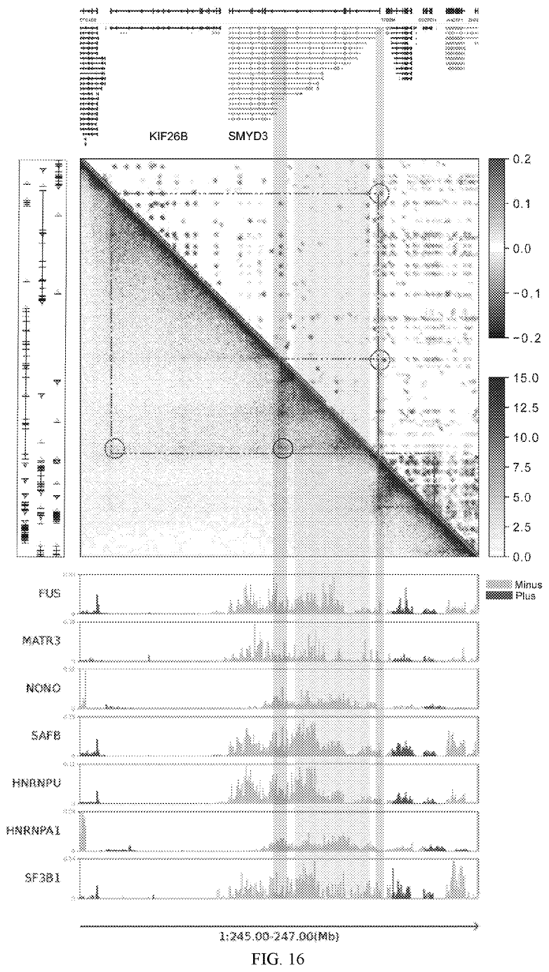

Further, in the visualization analysis of RNA and higher-order chromatin structures, the SMYD3 gene is used as an example to illustrate the promoting effect of lncRNA on specific chromatin loci and its potential molecular mechanism, further confirming that lncRNA actively regulates three-dimensional chromatin structure by mediating promoter-enhancer and CTCF-dependent chromatin interactions. In transcription factory regions, lncRNA does not participate in obvious chromatin interactions but may contribute to post-transcriptional processing and modification of nascent RNA by aggregating RBPs, as shown in FIGS. 15 and 16.

In summary, the present disclosure provides a method for enhancing chromatin interaction information based on RNA-DNA and DNA-DNA interaction information, the core of which is to introduce RNA as an endogenous regulator of chromatin spatial structure into chromatin interaction modeling and to accurately predict and enhance Hi-C maps using a deep learning model, thereby analyzing the specific regulatory effects of RNA on multi-scale chromatin hierarchical structures (e.g., chromatin loops, TADs, compartments).

Compared with existing technologies, the method of the present disclosure has the following advantages:
(1) Effective integration of RNA information into chromatin interaction modeling: By constructing a DNA-DNA feature correlation coefficient matrix integrating RNA-DNA interaction features, the present disclosure treats RNA as an “endogenous factor” in the regulation of chromatin conformation and, for the first time, systematically converts an non-symmetric RNA-DNA interaction matrix into a symmetric matrix embedded into chromatin analysis, overcoming the problem that RNA localization information cannot directly reflect DNA-DNA interactions.
(2) Improved spatial resolution of Hi-C maps by integrating multi-modal omics data: By introducing a dual-channel convolutional neural network-based deep learning model, the present disclosure combines RNA-DNA interaction information with Hi-C contact matrices to effectively enhance the interaction signals of Hi-C data, particularly showing stronger generalization ability and prediction accuracy under low-resolution or sparse data conditions, thus improving the modeling precision of chromatin spatial structure.
(3) Strong cross-RNA-type generalization ability: The prediction model of the present disclosure achieves stable prediction performance across multiple RNA types (e.g., lncRNA, mRNA, snRNA, snoRNA), enables analysis of the regulatory characteristics of different RNAs in chromatin structure, captures their functional differences across hierarchical structures (e.g., TADs, loops, compartments), and provides a theoretical basis for revealing the diversity of RNA regulatory mechanisms.
(4) Model attribution enabling quantitative functional analysis: The present disclosure provides an attribution analysis module that traces the source contribution of the model to specific chromatin structure predictions, thereby quantitatively evaluating the regulatory effects of different RNAs at key regions such as TAD boundaries and chromatin loop anchors. It reveals the dominant role of RNA in promoting chromatin loop formation and stabilization, as well as the potential mechanism of negative regulation at TAD boundaries, reflecting the unique advantages of this method in functional attribution and scale-dependent analysis.
(5) Visualization and cross-omics validation enhancing biological interpretability: The present disclosure combines CLIP-seq, ChIP-seq data, and subnuclear structure annotation to verify whether RNA-mediated chromatin loop anchors are enriched for RNA-binding proteins and epigenetic marks; it further visualizes the spatial enrichment patterns of different RNA types in chromatin regulation, yielding predicted results with higher biological interpretability and verifiability.
(6) Promoting refined development of research on 3D structure and function of chromatin: The present disclosure not only enables prediction and enhancement of Hi-C maps but also clarifies the regulatory patterns of RNA in promoter-enhancer interactions, nucleolar/nuclear speckle enrichment, and long-range chromatin associations via downstream analysis, providing key tools and methodological support for exploring the coupling between RNA-mediated chromatin structure and function.
(7) Broad applicability and scalability: The present disclosure is applicable to RNA-DNA interaction and Hi-C data from diverse cell types and public databases, exhibits good universality, and can be expanded to various bioinformatics applications such as epigenetic state prediction, regulatory element function identification, and RNA molecular function annotation.

It should be noted that in this document, the terms “comprise”, “include” or any other variants thereof are intended to cover a non-exclusive inclusion, such that a process, method, article, or terminal device that includes a series of elements not only includes those elements, but also includes other elements not expressly listed, or also includes elements inherent to such process, method, article, or terminal device. In the absence of further limitation, an element defined by the phrase “comprising a . . . ” does not exclude the presence of additional identical elements in the process, method, article, or terminal device that includes the element.

In the specification, reference to “one embodiment”, “an embodiment”, “an exemplary embodiment”, “some embodiments” and the like indicates that the embodiment described may include a particular feature, structure, or characteristic, but not every embodiment necessarily includes that particular feature, structure, or characteristic. Moreover, when a particular feature, structure, or characteristic is described in connection with an embodiment, it is within the knowledge of a person skilled in the relevant art to implement such feature, structure, or characteristic in connection with other embodiments (whether explicitly described or not).

It should be understood that the term “and/or” herein is merely an association relationship describing associated objects, indicating that three relationships may exist. For example, A and/or B may indicate: A exists alone, both A and B exist, or B exists alone, where A and B may be singular or plural. In addition, the character “/” herein generally indicates that the associated objects are in an “or” relationship, but may also indicate an “and/or” relationship, which may be understood in context.

In the present disclosure, “at least one” means one or more, and “a plurality of” means two or more. The expression “at least one of the following” or a similar expression means any combination of these items, including any combination of a single item or plural items. For example, at least one of a, b, or c may mean: a, b, c, a-b, a-c, b-c, or a-b-c, where a, b, c may be single or multiple.

It should be understood that in various embodiments of the present disclosure, the sequence numbers of the above processes do not imply an order of execution, and the execution order of the processes should be determined by their functions and internal logic, and should not constitute any limitation on the implementation of the embodiments of the present disclosure.

In the several embodiments provided in the present disclosure, it should be understood that the disclosed devices, apparatuses, and methods may be implemented in other manners. For example, the apparatus embodiments described above are merely illustrative. For example, the division of units is merely a logical functional division, and in actual implementation there may be other division manners, for example, multiple units or components may be combined or integrated into another device, or some features may be omitted or not performed. In addition, the mutual coupling or direct coupling or communication connection shown or discussed may be indirect coupling or communication connection through some interfaces, devices, or units, and may be electrical, mechanical, or other forms.

The units described as separate components may or may not be physically separate, and the components shown as units may or may not be physical units, i.e., they may be located in one place or distributed over multiple network units. Some or all of the units may be selected according to actual needs to achieve the objectives of the technical solutions of the embodiments.

In addition, the functional units in the various embodiments of the present disclosure may be integrated into one processing unit, or each unit may exist physically separately, or two or more units may be integrated into one unit.

If the functions are implemented in the form of software functional units and sold or used as independent products, they may be stored in a computer-readable storage medium. Based on such understanding, the technical solutions of the present disclosure, essentially or the part contributing to the existing technology, or part of the technical solutions, may be embodied in the form of a software product. The computer software product is stored in a storage medium and includes several instructions for causing a computer device (which may be a personal computer, a server, or a network device, etc.) to perform all or part of the steps of the methods described in the various embodiments of the present disclosure. The aforementioned storage medium includes: a USB flash drive, a removable hard disk, a read-only memory (ROM), a random access memory (RAM), a magnetic disk, or an optical disk, and other media that can store program codes.

The present disclosure covers any alternatives, modifications, equivalent methods, and solutions that are within the spirit and scope of the present disclosure. In order to provide the public with a thorough understanding of the present disclosure, specific details are described in detail in the preferred embodiments of the present disclosure, and those skilled in the art can fully understand the present disclosure without the description of these details. In addition, well-known methods, processes, processes, components, and circuits are not described in detail to avoid unnecessarily obscuring the essence of the present disclosure.

The above descriptions are merely preferred embodiments of the present disclosure and are not intended to limit the present disclosure. Any modifications, equivalent replacements, improvements, etc., made within the spirit and principles of the present disclosure shall be included within the protection scope of the present disclosure.

What is claimed is:

1. A method for enhancing chromatin interaction information based on Ribonucleic Acid (RNA)-Deoxyribonucleic Acid (DNA) and DNA-DNA interaction information, comprising the following steps:
S1, collecting in situ Mapping of RNA-Genome Interaction (iMARGI) data of RNA-DNA interactions and raw High-throughput Chromosome Conformation Capture (Hi-C) data of DNA-DNA interactions from the same cell line;
S2, performing feature extraction on the iMARGI data, and then calculating a Pearson correlation coefficient, to obtain a genome-wide DNA-DNA feature correlation coefficient matrix integrating RNA information; performing downsampling on the raw Hi-C data, and then performing Filtering by Adaptive Normalization (FAN) completion processing to obtain a completed Hi-C matrix;
wherein step S2 specifically comprises:
performing feature extraction on the RNA-DNA interaction data, and performing Pearson correlation coefficient calculation on the extracted features, to obtain the DNA-DNA feature correlation coefficient matrix integrating RNA information;
performing Knight-Ruiz (KR) normalization processing on the raw Hi-C data, performing smoothing processing on the KR-normalized Hi-C data using downsampling rates of 1/25 and 1/100, and performing completion processing on the downsampled Hi-C data using the FAN method to obtain the completed Hi-C matrix;
S3, constructing a Convolutional Neural Network (CNN) network model, taking the DNA-DNA feature correlation coefficient matrix integrating RNA information and the completed Hi-C matrix as inputs of the model, and training the model, wherein an output of the model is an enhanced Hi-C matrix;
wherein in step S3, for the completed Hi-C matrix, performing downsampling twice using a Double_Conv module combined with a DownConv layer, performing upsampling twice using the Double_Conv module combined with an UpConv layer, and finally obtaining a first result via the Double_Conv module; for the DNA-DNA feature correlation coefficient matrix integrating RNA information, obtaining a second result using the Double_Conv module three times; concatenating the first result and the second result, and then obtaining a final enhanced Hi-C matrix using the Double_Conv module twice;
S4, for the trained model, inputting data of different RNA types to predict a corresponding Hi-C matrix for downstream analysis.

2. The method according to claim 1, wherein step S1 specifically comprises:
obtaining the iMARGI data and the raw Hi-C data from public databases or experimental data; performing quality control on the obtained raw data to remove noisy data.

3. The method according to claim 1, wherein in step S3, the inputs of the model comprise two parts: the DNA-DNA feature correlation coefficient matrix integrating RNA information and the completed Hi-C matrix;
wherein sample division is performed in the following manner: dividing the DNA-DNA feature correlation coefficient matrix integrating RNA information and the completed Hi-C matrix into n K×K dimensional sub-matrices as input samples; and
dividing the input samples into a training set, a validation set, and a test set, which are respectively used for training, validation, and evaluation of the model.

4. The method according to claim 1, wherein in step S3, an error between a predicted Hi-C matrix and a ground-truth Hi-C matrix is measured by a loss function;
Least Absolute Deviations Loss (L1 loss) and perceptual loss are combined to optimize model weights, to handle data imbalance and improve performance of the model on sparse data;
an Adaptive Moment Estimation (Adam) optimizer is used to train the model, and cross-validation and parameter tuning are performed to improve generalization ability of the model.

5. The method according to claim 1, wherein step S3 further comprises:
applying the model to data of different RNA types, wherein an input is preprocessed RNA data, and an output is a predicted chromatin interaction enhancement matrix which is an enhanced Hi-C matrix; verifying prediction accuracy and stability of the model by comparing the enhanced matrix with a ground-truth Hi-C matrix.

6. The method according to claim 1, wherein in step S4, the downstream analysis comprises: Hi-C chromatin loop analysis mediated by different RNA types, validation using Cross-Linking and Immunoprecipitation Sequencing (CLIP-seq) data and Chromatin Immunoprecipitation Sequencing (ChIP-seq) data, enrichment of chromatin loops in subnuclear structures and Enhancer-Promoter (EP) sites, effects of different RNA types on chromatin hierarchical structures, exploration of effects of RNA on chromatin conformation via model attribution, exploration of effects of RNA on Topologically Associating Domain (TAD) boundaries and chromatin loop anchors via model attribution, and visualization analysis of RNA and higher-order chromatin structures.

7. The method according to claim 6, wherein in the downstream analysis, the Hi-C chromatin loop analysis mediated by different RNA types comprises: identifying chromatin loops on raw Hi-C data and predicted Hi-C data using a Mustache tool, and performing Venn analysis on the chromatin loops of the raw data and the chromatin loops of the predicted data;
the validation using CLIP-seq data and ChIP-seq data comprises: evaluating, using CLIP-seq data, whether, near chromatin loop anchors mediated by different RNA types, there is specific co-localization with RNA-binding proteins (RBPs) bound to nascent RNA; analyzing, in combination with public ChIP-seq data, whether chromatin loop anchors in predicted results are enriched for additional chromatin regulatory factors;
the enrichment of chromatin loops in subnuclear structures and EP sites comprises: respectively calculating enrichment of Hi-C data predicted by different types of RNA in subnuclear structures and EP sites, revealing that long non-coding RNA (lncRNA) frequently participates in construction of promoter-enhancer interactions and that small nuclear RNA (snRNA) and small nucleolar RNA (snoRNA) play roles in nucleolus and nuclear speckle regions;
the effects of different RNA types on chromatin hierarchical structures comprise: respectively calculating effects of different types of RNA on chromatin loops, TADs, and A/B compartments of Hi-C data, revealing that lncRNA participates in local fine regulation, while snRNA/snoRNA has advantages in long-range interactions and larger-scale structural remodeling.

8. The method according to claim 6, wherein in the downstream analysis, the exploration of effects of RNA on chromatin conformation via model attribution comprises: calculating contribution features of lncRNA, messenger RNA (mRNA), snRNA, and snoRNA at TAD boundaries and chromatin loop anchors, revealing a promoting effect of RNA on chromatin loop structures and negative or neutral regulation at TAD boundaries, indicating that the effects of RNA on chromatin conformation are scale-dependent and tend to mediate small-scale, specific chromatin loop structures;
the exploration of effects of RNA on TAD boundaries and chromatin loop anchors via model attribution comprises: calculating contribution features of lncRNA, mRNA, snRNA, and snoRNA at TAD boundaries and chromatin loop anchors, and displaying interaction distribution maps with positional information for chromatin interactions at a given locus affected and unaffected by RNA.
